(19) United States
(12) Patent Application Publication
(10) Pub. No.: US 2008/0124318 A1
(43) Pub. Date: May 29, 2008

(54) Algae supplement and treatment method

(76) Inventor: Jerold Lisson, Henrietta, NY (US)

Correspondence Address: LAW OFFICE OF TRACY P. JONG, 2775 BUFFALO ROAD, SUITE 3, ROCHESTER, NY, 14624, US

(21) Appl. No.: 11/800,018

(22) Filed: May 3, 2007

Related U.S. Application Data
(60) Provisional application No. 60/861,462, filed on Nov. 28, 2006.

(51) Int. Cl. A61K 35/74; A61K 39/395; A61P 39/00

(52) U.S. Cl. 424/130.1; 424/93.4

(57) ABSTRACT

This invention relates to a method and composition for enhancing pluripotent cell production, longevity and homing to injured or diseased sites, and more specifically, by administration of a therapeutic dosage of cyanobacteria and green algae. The increased concentration of pluripotent cells within the circulatory system of animals is effected by the transient increase of pluripotent cell production and the concommitant extension of the pluripotent cell life cycle.

### PRIORITY CLAIM AND RELATED APPLICATIONS

This application claims the benefit of priority from provisional application U.S. Ser. No. 60/861,462 filed on Nov. 28, 2006 and provisional application U.S. Ser. No. 60/903068 filed on Feb. 23, 2007 in the name of Jerold Lisson of 61 Blackwell Lane, Henrietta, N.Y. Each of said applications is incorporated by reference in its entirety.

### FIELD

This invention relates to a method and composition for enhancing pluripotent cell production, longevity and homing to injured or diseased sites, and more specifically, by administration of a therapeutic dosage of cyanobacteria and green algae.

### BACKGROUND

Two concomitant factors that affect the health or well being of higher ordered animals, such as humans and other mammals, are the ability to regenerate damaged tissue and the management of disease. It has been observed that some primitive cultures who supplement their diet with green algae appear to be healthier and experience longer life spans than western civilizations. They observed a much speedier recovery from illnesses, faster healing of wounds, and an apparent slower aging process. Researchers acknowledge much of this may be due to socio-psycho influences on their life style and the very limited exposure to western diseases. However, they also observed many differences in their dietary habits. One notable difference was the use of cyanobacteria and green algae in their diet.

Research has shown that the cyanobacteria produces and stores large quantities of chemicals in the L-selectin ligand family. These chemicals are known to play a key role in slowing or ceasing the aging process of pluripotent cells. Thus, Applicant believes that the use of specific species of cyanobacteria and/or green algae (or their respective extracts) increase the lifetime and activity of available pluripotent cells via the antioxidant's protective mechanisms. This, in turn, allows for the faster replacement of damaged tissue and consequently reduces the risk of pathogens colonizing on damaged tissue. Organs and tissues remain healthier as the replacement of aged cells is quicker with the increased pluripotent cell availability, slowing down the natural aging process. Reference may be had to, e.g., “Neutraceuticals Promote Proliferation of Human Stem Cells” by Paula Bickford, et al., published in Stem Cells and Development vol. 15 (2006) and “Blueberry- and spirulina-enriched diets enhance strital dopamine recovery and induce a rapid, transient microglia activation after injury of the rat nigrostriatal dopamine system” by Ingrid Stromberg, et al., published in Experimental Neurology 196 (2005) 298-307.

U.S. Pat. No. 6,814,961 discloses a method to enhance trafficking or homing of stem cells by administering a therapeutically effective amount of blue-green algae to an animal subject. The compounds described therein are effective, however, do not contemplate the synergistic effects of additional potent antioxidant components that increase the activity of and prolong the life cycle of the pluripotent cells. Additionally, Inventor-Applicant believes that his novel composition incorporates a greater concentration of pluripotent cell enhancing cyanobacteria than is commercially available in supplement form.

The cyanobacteria and green algae are a remarkable storehouse of major nutrients that are synergistic in their natural form. The novel algae composition of this invention has superlative regenerative and prophylactic power via an optimal mix of two to four inter-supporting and synergistic green algae and cyanobacteria components. This interaction eliminates the need for multiple food sources currently consumed in the average diet. Therapeutic action of astaxanathin of the green algae includes antioxidant properties and inhibiting the catabolic effects of heavy exercise by muscle tissue cellular oxidation. Applicant believes the simultaeous synergy of the potent antioxidant action and pluripotent cell production provides an improvement over the prior art.

Similarly, Applicant believes that well functioning body systems will be more receptive to, and benefit more from, the regeneration processes offered by the additional pluripotent cell production. By combining cyanobacteria with green algae components, potent antioxidant activity enhances the general health of the subject and inhibits the harmful effects of disease causing organisms, thus the regenerative process benefits will be amplified.

Stem cells are pluripotent cells derived from stromal or basal tissue capable of differentiating into more specialized cells. Stem cells, from both stromal and basal tissue, have been found to differentiate into a variety of non-hematopoietic tissue specific cell types, such as myocytes, heptocytes, osteocytes, glial cells, epithelial cells and neurons. Hematopoietic stem cells can also differentiate into many different types of blood cells including red blood cells, platelets and leukocytes. Hematopoietic stem cells are quite abundant and play a role in the continuous lifelong replenishment of blood cells.

U.S. Pat. No. 6,814,961 discloses that a therapeutically effective amount of blue-green algae induces a transient increase in the population of some stem cells, such as CD34+ stem cells, in the subject's circulatory system. More specifically, the administration of a polysaccharide rich fraction of whole AFA increases the homing of CD34+ and NK cells from the circulatory system to various parts of the body. The entire disclosure of said patent is incorporated by reference herein. The percentage increase in the number of circulating stem cells, compared to a control, is ranges from about 10% to more than about 500%, following administration of the blue-green algae. U.S. Pat. No. 6,814,961 further discloses studies that determined the circulating levels of CD34+ stem cells decreased to near pre-existing levels within 4 hours.

The prophylactic use of natural antioxidants to increase the lifetime of cells in mammals has been well recognized. The antioxidants react with toxins (free radicals) either in the circulatory system or at cell site to inactivate the toxins prior to damaging the cells. U.S. Pat. No. 7,074,990 describes the role of specific caroteniods in the lifetime extension of pluripotent cells. U.S. Pat. No. 6,884,783 describes the use of other antioxidants such as 7-hydoxy chromones for enhancing the lifetime of pluripotent cells. U.S. Pat. No. 7,025,965 describes the use of phycoicyanins from blue green algae for the treatment of inflammation by reacting with toxins causing cell destruction. The entire disclosure of each of said patents is incorporated by reference herein.

Algae is rich in many antioxidants such as beta-carotene, astaxanthin, phycoicyanins, 7-hydoxy chromones and a plurality of unspecified antioxidants. Some of these antioxidants comprise polysaccharides and components thereof. Many of these antioxidants are specific to certain pluripotent cells (e.g. 7-hydoxy chromones).

The novel compositions of this invention are advantageous over prior art nutritional supplements in two important ways. First, the synergy between the pluripotent cell enhancing components and the antioxidant components yields unexpected results that are more than the benefits of the sum of each component individually. Secondly, the paucity of ingredients provides for a nutritional supplement that is both simpler and lower cost to manufacture. Additionally, with fewer ingredients, there is less liklihood of interactions among the various components of the composition and less liklihood of negative reactions (e.g., drug interactions, allergic reactions, side effects, and the like) for the subject. Not only are there fewer components, but the prophylatic, therapeutic and regenerative properties of the components are superior to those utilized in prior art supplements and allow for custom blending of the components as medically indicated or desirable for each individual subject.

The Applicant has found that a novel algae composition comprising a powdered form of whole plant extracts of cyanobacteria and at least one green algae as active components provides enhanced stamina and endurance in motor function, for example, by prolonging the ability to perform repetitive movement and delaying the onset of muscle fatigue. The novel algae composition of this invention also improves general health.

Thus, it is desirable to provide a compound that enhances pluripotent cell (e.g., bone marrow stem cell) production, enhances its emission into the bloodstream, increases the activity of the pluripotent cells, prolongs the life cycle of the pluripotent cells, enhances the homing of the pluripotent cells to damaged or stressed tissues, and optimizes the general physiological condition of the subject so the beneficial regenerative effects can be realized by absorption in said stressed tissues.

### SUMMARY

A method is disclosed herein for enhancing health and disease control in a subject, comprising: administering to a subject a composition comprising a therapeutic dosage comprising green algae and cyanobacteria. In one embodiment, the therapeutic administration of the novel composition of this invention induces the increase of circulating transient puripotent cells by as much as 500%.

A method is disclosed herein for enhancing the production of pluripotent cells and increasing the longevity of said pluripotent cells in a subject, comprising: administering to a human a composition comprising a therapeutic dosage comprising green algae and cyanobacteria, thereby enhancing pluripotent cell concentration in a subject. In one embodiment, the antioxidant activity in the novel composition of this invention induces the longevity of pluripotent cells, and thus an increase of circulating transient puripotent cells, by more than about 4 hours, by more than about 6 hours, by more than about 8 hours, by more than about 12 hours, by more than about 24 hours, by more than about 36 hours, by more than about 48 hours, and by more than about 72 hours. This can dramatically increase the effectiveness of the pluripotent cells in treating disease both at normal levels and the increased levels during the transient period.

A composition is disclosed herein for enhancing health and disease control in humans, comprising: a therapeutic dosage comprising green algae and cyanobacteria. In one embodiment, said green algae comprises a green algae selected from the group consisting of Haematocccus pluvialis, Chlorella vulgaris, Chlorella pyrenoidosa, Dunaliella salina or a combination thereof. In one embodiment, said cyanobacteria comprises a cyanobacteria selected from the group consisting of Aphanizomenon flos-aquae, Spirulina pacifica, Spirulina plantesis or a combination thereof. In a preferred embodiment, said composition comprises a form selected from the group consisting of a dry powder, liquid suspension and tablet. The novel algae composition of this invention provides therapeutic and prophylactic health benefits in the nature of protection of cellular tissues from the degenerative processes of free radicals and aging.

In one embodiment, a novel algae composition of this invention further comprises inert ingredients such as pharmaceutically acceptable carriers, texture enhancers and palatability enhancers. In a preferred embodiment, said palatability enhancer comprises xylitol.

In some embodiments, the subject provided the treatment is healthy. In other embodiments the subject has known disease or physiological disorders.

The Applicant has found that a novel algae composition comprising a powdered form of whole plant extracts of cyanobacteria and at least one green algae as active components provides enhanced stamina and endurance in motor function, for example, by prolonging the ability to perform repetitive movement and delaying the onset of muscle fatigue. The novel algae composition of this invention also improves general health.

The novel algae composition of this invention may be orally administered therapeutically or prophylatically, or most preferably, in a therapeutic and prophylatic combination. The treatment is most effectively delivered in multiple doses at regular intervals or via a slow release device or a depot. In some embodiments, the subject provided the novel algae composition is healthy. In other embodiments, the subject suffers a disease or physiological condition. In some embodiments, the subject is a human subject. In other embodiments, the subject is a veterinary subject, preferably a multicellular, vertebrate organism including, for example, mammals.

It is an object of this invention to provide an algae composition which enhances pluripotent cell production.

It is an object of this invention to provide an algae composition which enhances the life cycle of pluripotent cells.

It is an object of this invention to provide an algae composition which enhances the activity of pluripotent cells.

It is an object of this invention to provide an algae composition which treats general symptoms of aging and depressed tissue regeneration processes.

It is an object of this invention to provide an algae composition which provides both protective and regenerative health benefits.

It is an object of this invention to provide an algae composition which enhances mental well-being and/or attitude, mental focus and provides an anti-depressant effect.

It is an object of this invention to provide an algae composition which treats depressed tissue regeneration processes resulting from injury, disease or illness.

It is an object of this invention to provide an algae composition which increases stamina and endurance in motor function, for example, by prolonging the ability to perform repetitive movement and delaying the onset of muscle fatigue.

It is an object of this invention to provide an algae composition which may be delivered in more palatable (tasteful) and desirable manners and food products than those currently available to consumers.

It is an object of this invention to provide an algae composition which may be selectively modified by the user within prescribed limits to produce desired nutritional content, health results or treatment regimen.

It is yet another object of this invention to provide a novel algae composition that is economical for mass production from the viewpoint of the manufacturer and consumer, thereby making it economically available to the buying public.

Whereas there may be many embodiments of the present invention, each embodiment may meet one or more of the foregoing recited objects in any combination. It is not intended that each embodiment will necessarily meet each objective.

Thus, having broadly outlined the more important features of the present invention in order that the detailed description thereof may be better understood, and that the present contribution to the art may be better appreciated, there are, of course, additional features of the present invention that will be described herein and will form a part of the subject matter of the invention.

In this respect, before explaining at least one embodiment of the invention in detail, it is to be understood that the invention is not limited in its application to the details of construction and the arrangements of the components set forth in the following description. The present invention is capable of other embodiments and of being practiced and carried out in various ways. Also it is to be understood that the phraseology and terminology employed herein are for the purpose of description and should not be regarded as limiting.

### DEFINITIONS

As used in this specification, stem cell means a pluripotent, self-maintaining cell that gives rise to progeny of many tissue types, including but not limited to the entire hematopoietic and bone marrow stromal cell lineages. A typical endogenous stem cell resides in the bone marrow, although stem cells also reside in the intestinal epithelium, tongue mucosa, epidermis, testis, and the like. It is to be understood that the invention is described with reference to primarily bone marrow stem cells, but that it is intended to apply to these other stem cell types as well.

As used in this specification, cyanobacteria means a gram-negative photosynthetic bacteria belonging to Division Cyanophyta that may exist in unicellular, colonial, or filamentous forms. By way of example, but not limitation, cyanobacteria may include, but are not limited to, Spirulina species, aphanizomenon species, Aphanizomenon flos aquae (AFA), and the like. In a preferred embodiment, cyanobacteria comprises an L-selectin ligand. Cyanobacteria may also be known as blue-green algae.

As used in this specification, green algae means photosynthetic paraphyletic organism, a eucaryote with organelles that may exist in unicellular, diatomic, colonial, or filamentous forms. By way of example, but not limitation, green algae may include, but are not limited to, Chlorella species (e.g., Chlorella vulgaris, Chlorella pyrenoidosa and the like), Haematoccoccus pluvialis (H. pluvialis) species, Dunaliella species, and the like. In a preferred embodiment, green algae comprises astaxanathin.

As used in this specification, algae means any fraction, extract, or isolated or purified molecule from an algae cell. In one embodiment, the component is a protein or nucleic acid. In another embodiment, the component is a phytochemical. In another embodiment, the component is a fraction of algae. Extracts may be prepared according to the teachings of U.S. Pat. No. 6,977,076 (methods for obtaining from Chlorella extract polysaccharides having immunomodulatory properties), U.S. Pat. No. 6,814,961 (method for enhancing stem cell trafficking), and the like.

As used in this specification, cyanobacteria means any fraction, extract, or isolated or purified molecule from an cyanobacteria cell. In one embodiment, the component is a protein or nucleic acid. In another embodiment, the component is a phytochemical. In another embodiment, the component is a fraction of cyanobacteria. Extracts may be prepared according to the teachings of U.S. Pat. No. 6,977,076 (methods for obtaining from Chlorella extract polysaccharides having immunomodulatory properties), U.S. Pat. No. 6,814,961 (method for enhancing stem cell trafficking), and the like.

As used in this specification, Spirulina refers to Arthrospira (Spirulina) platensis and/or Spirulina pacifica.

As used in this specification, Chlorella refers to a genus of water-grown unicellular green algae belonging to the Phylum Chlorophyta.

As used in this specification, effective amount means an amount of compound or mixture capable of activating or enhancing pluripotent cell production that can be determined by various methods used in the biological sciences, including generating an empirical dose-response curve. In one embodiment, a “therapeutically effective amount” is an amount effective for enhancing production of pluripotent cells that replenish, repair, or rejuvenate tissue. A therapeutically effective amount also may be an amount sufficient for treating a condition or disease.

As used in this specification, administration to a subject means oral or parenteral administration of the novel compound in the form of a unit dose in solid, semi-solid, or liquid dosage form such as tablets, pills, powders, gels, pastes, capsules, coated tablets, liquid solutions, or liquid suspensions.

As used in this specification, tissue means any organized mammilian cell structure, such as, but not inclusive of epithelium, muscle, liver, lung or bone.

As used in this specification, disease management means the destruction of non organized pathogens (destructive bacteria, parasites, or viruses) as they enter the human system via open wounds, ingestion, breathing and the like and the destruction of colonized pathogens in the form of wound abscesses, pneumonia, intestinal infections, and the like. These colonized pathogens may be in any tissue or body fluid and may be caused by an undefined species of pathogens.

As used in this application, a toxin means any chemical that either reacts with the plasma membrane or cytoplasm to prevent cell respiration or metabolism. By way of illustration, but not limitation, toxins may be oxidants or free radical generators.

As used in this application, immunoglobulin means a polysaccaride on the membrane surface or in the fluidic system of a cell whose function is to intercept and elminate toxins.

As used in this application, active ingredient is any component comprising either a chemical moiety, cells or cell extract that is necessary for the therapeutic regime of a subject.

As used in this application, inert ingredient means any non-therapeutic material added to the mixture which does not contribute to the generation, homing or lifetime enhancement of pluripotent cells and may include, but is not limited to, flavorings, sweeteners, surfactants, solid bulk diluents, binders, pharmaceutically acceptable carriers, texture enhancers, palatability enhancers, preservatives and the like.

As used in this application, pharmaceutically acceptable carrier means any bulk medium that the algae and cyanobacteria are add to for the proper administration of the algae and cyanobacteria. They may include water, lipids and/or. These also may contain adjunct materials such as salts, surfactants, buffers, thickeners and preservatives.

As used in this application, texture enhancer means any bulk diluent added to the mixture to improve consistency such that it may be administered orally to the subject.

As used in this application, palatability enhancer means materials added to the mixture to make oral administering of the algae and cyanobacteria more pleasant to the subject. These may include flavorings and sweeteners.

As used in this application, circulatory system means the structure that moves blood and blood components throughout the body of animals, including the heart, blood vessels and lymph vessels.

As used in this application, diseased site means a site in an animal where nonconforming cells are present. These nonconforming cells may either be pathogens, such as bacteria and viruses, or abnormal cells such as carcinomas.

As used in this application, injured site mans any tissue that is damaged either by mechanical, chemical or bacterial means.

As used in this application, enhanced concentration site means any site where the concentration of pluripotent cells is elevated above the concentration measured at normal conditions.

As used in this application, antioxidant means any chemical moiety that reacts with chemicals containing free radicals, reducing their capability to react/oxidize bioactive chemicals in cells.

As used in this application, pluripotent cells are nondifferentiated stem or daughter cells whose progeny produce specific tissues such as blood, bone, neuron, epithelial and the like.

As used in this application, liquid suspension is any uniform mixture consisting of a liquid containing undissolved solids.

As used in this application, beta-CDS is the scientific identifier for a super family of Immunoglobulins.

As used in this application, environmental toxin is any water or airborne chemical or biological moiety which will cause negative cell function.

As used in this application, tablet means a solid form single dosage such as gel caplet, pill, capsule, flake or the like.

### DETAILED DESCRIPTION

The present invention concerns a method and composition for enhancing health and disease control in a subject. In one embodiment of the novel method of this invention, the method comprises administering to a subject a composition comprising a therapeutic dosage comprising green algae and cyanobacteria. In one embodiment, the therapeutic administration of the novel composition of this invention induces the increase of circulating transient puripotent cells by as much as 500%.

In one embodiment, the subject is a mammal. In one embodiment, the subject is human.

In one embodiment of the novel method of this invention, the method for enhancing the production of pluripotent cells and increasing the longevity of said pluripotent cells in a subject comprises administering to a human a composition comprising a therapeutic dosage comprising green algae and cyanobacteria, thereby enhancing pluripotent cell concentration in a subject. In one embodiment, the antioxidant activity in the novel composition of this invention induces the longevity of pluripotent cells, and thus an increase of circulating transient puripotent cells and the concentration at enhanced concentration sites, by more than about 4 hours, by more than about 6 hours, by more than about 8 hours, by more than about 12 hours, by more than about 24 hours, by more than about 36 hours, by more than about 48 hours, and by more than about 72 hours. This can dramatically increase the effectiveness of the pluripotent cells in treating disease both at normal levels and the increased levels during the transient period.

In one embodiment of the novel composition of this invention, the composition for enhancing health and disease control in humans comprises a therapeutic dosage comprising green algae and cyanobacteria. In one embodiment, said green algae comprises a green algae selected from the group consisting of Haematocccus pluvialis, Chlorella vulgaris, Chlorella pyrenoidosa, Dunaliella salina or a combination thereof. In one embodiment, said cyanobacteria comprises a cyanobacteria selected from the group consisting of Aphanizomenon flos-aquae, Spirulina pacifica, Spirulina plantesis or a combination thereof. In a preferred embodiment, said composition comprises a form selected from the group consisting of a dry powder, liquid suspension and tablet. The novel algae composition of this invention provides therapeutic and prophylactic health benefits in the nature of protection of cellular tissues from the degenerative processes of free radicals and aging.

In one embodiment, a novel algae composition of this invention further comprises inert ingredients such as pharmaceutically acceptable carriers, texture enhancers and palatability enhancers. In a preferred embodiment, said palatability enhancer comprises xylitol.

This potential therapy option for various pathologies focuses on the body's own self healing mechanisms rather than invasive surgeries or pharmacological treatment regimens with potential adverse side effects and drug interactions. The aging population, the increasing demand for active retirement lifestyles and quality of life, and the ever increasing costs of medical care provide a climate for serious consideration of these alternative and holistic alternatives.

The health benefits of cyanobacteria and green algae, as well as their antioxidants, are acknowledged through historical usage. However, the use of each independently has limited value to the recipient. The increased number of pluripotent cells would be of little value if they were quickly destroyed by oxidants. Similarly, the use of antioxidants would be of marginal value where there existed only an insignificant number of living pluripotent cells due to aging processes. The combination of these natural products, the cyanobacteria and green algae, has a synergetic effect. Chemicals and/or components in the cyanobacteria assist in increasing the concentration of and/or extending the lifetime of the pluripotent cells. At the same time, the antioxidants in the green algae assist in the protection of these cells from the ravaging effects of oxidation and/or extending the pluriponent cell lifetime.

In one embodiment, it is the increased combination of the algae in the novel composition, as compared to other commercially available supplements, that induces these results. While the dietary supplement of the present invention may be used by anyone who wishes to enhance their pluripotent cell production/lifetime/homing/activity, physical stamina and endurance in motor function, or general health, the algae supplement of the present invention is particularly beneficial for elderly people, infirmed people and athletes where natural tissue regeneration functions may be depressed or improved tissue regeneration capabilities are necessary. Each of the constituent algae and microbial components has an individual tendency to enhance physical well being. However, the combination of cyanobacteria (e.g., AFA) and a green algae (e.g., H. pluvialis) (and optionally with other algae extracts and natural supporting components), when administered in proper concentration, stimulates pluripotent cell production/lifetime/homing/activity and physical stamina and endurance in motor function. The synergism between all of the components render the administration of a combination composition containing each algae and microbial component desirable.

In one embodiment, the pluripotent cell concentration is increased at the enhanced concentration site. Such enhanced concentrations are the circulatory system, a diseased site or an injured site.

In a preferred embodiment, the novel algae composition of this invention further comprises a second species of cyanobacteria and a second species of green algae. The primary active components are antioxidants. The inclusion of additional antioxidant components permits the user to selectively optimize the ratio of algae to antioxidant to obtain a desired effect or synergism for a particular treatment regimen.

Applicant believes that whole foods provide the optimum nutritional sources. As opposed to purified or isolated vitamins and minerals, or concentrated vitamin compositions, whole foods contain supporting elements that synergistically enhance the individual activity of each component—that is, the micro-ecosystem has the optimum balance of ambient proteins, fats, saccharides, vitamins, minerals and the like to achieve the highest activity levels of each individual component. Thus, whole plant components are preferably used in the manufacture of the novel algae composition of this invention. Although less preferable, synthesized components and fractional components of the whole algae may also be used in the instant invention.

In one embodiment, the cyanobacteria and/or green algae provide enhanced concentration of certain trace minerals.

Different compositions of the invention are contemplated, as well as different methods of delivery and administration to the patient. By way of illustration, but not limitation, the invention may be formed into a pill (e.g., tablet), dry powder, topical cream, liquid suspension or oral spray according to the teachings of U.S. Pat. No. 6,586,018 (algae composition), U.S. Pat. No. 7,081,259 (algae, extract having therapeutic activity on injuries, and pharmaceutical composition and health food containing the same), U.S. Pat. No. 6,852,344 (algae composition for treating CD34+ acute and chronic myeloid leukemia and a method thereof), and the like. The entire disclosure of each of said patents is hereby incorporated by reference into this specification. The novel composition of this invention may also be formed into a freeze dried product, liquid, oleoresin or supercritical fluid extraction. Additionally, as will be described in more detail below, a powder or liquid nutritional supplement mixture may be formed for prepackaged distribution and mixed directly with food products by the user.

### Pluripotent Cells and Regenerative Processes in Living Organisms

Pluripotent cells play a role in the continuous lifelong physiological replenishment, healing and regenerative processes of various tissues and organs in living organisms. Pluripotent cells are capable of differentiating into more specialized cells such as myocytes, hepatocytes, osteocytes, glial cells, neurons, fibroblast or fibroblast-like cells, liver cells, myocardium cells, epithelial cells, brain cells, organ cells, and the like. Hematopoietic stem cells can also differentiate into many different types of blood cells including red blood cells, platelets and leukocytes. Hematopoietic stem cells are quite abundant and play a role in the continuous lifelong replenishment of blood cells.

Therefore, activation and enhancement of pluripotent cell production can provide regenerative mechanisms for tissue cells that are at the end of their life cycle, damaged or stressed. The beneficial effects are further optimized by enhanced homing of the stem cells and simultaneous stimulation of supporting systems such as the circulatory system, respiratory system, nervous system, gastrointestinal system, and the like.

The expeditious replacement of damaged tissue is critical to the return of the organ or tissue to its normal function and the prevention and/or reduction of the colonization of unwanted pathogens. The availability of on-site pluripotent cells (e.g., stem, stromal or basal cells) are a mandatory part of the replacement process. The rate of replacement is dependent upon many factors. These may include, but are not limited to, the ability and/or signaling process to induce the pluripotent adult stem cell (i.e. basal or stromal) to produce “daughter” cells, the condition of the circulatory system to move the pluripotent cells and/or nutrients to the appropriate site, and the availability of active pluripotent cells.

Pluripotent cell lifetime is an important aspect of the replacement rate of tissue. Many stem cells do not contain the ability to clone themselves. Thus, when they die or become inactive, reproductive function at that site is limited or fails completely. One demonstrative example might be baldness. As basal cells in the hair follicles die, hair regeneration ceases and one becomes bald. In the case of non-existent pluripotent cells, only transplantation would be an effective treatment.

Much research has been accomplished and is on-going to better understand factors that determine the lifetime of various pluripotent cells. While the exact nature of the aging process is not well understood, it has been determined that the lifetime of certain pluripotent cells (e.g. stem cells) may be extended by chemical treatment. Research has demonstrated the use of natural products such as cyanobacteria and green algae may be helpful in discrete cases as they contain many beneficial chemicals. Some research suggests that beneficial chemicals in cyanobacteria and green algae may include astaxanthin, L-selectin ligands, potent antioxidants, and the like. Potent antioxidants may include, for example, astaxanthin, zeaxthanin, 9-cis-beta-carotene and beta-CDS. The algae stimulates cytokines and interleukins.

Another factor for determining the lifetime of pluripotent cells is the susceptibility and/or rate of attack by detrimental chemical toxins. These may be in the form antibodies, enzymes, or just “toxic” chemicals. The oxidation process may either denature specific plasma proteins and restrict the osmosis of essential nutrients into the cell or interfere with the metabolic cell processes. In either case, eventually the cell ceases to function. Sources of these oxidants (free radical generators) include, but are not limited to, ingestion and/or metabolism byproducts of the host or parasitic microorganisms. These oxidants are often specific to the cell type or cytoplasm they attack. The use of antioxidants to reduce the destruction of cells has been well demonstrated in scientific literature.

The use of antioxidants (free radical scavengers) can extend the useful lifetime of pluripotent cells. These antioxidants can either be free floating in the circulatory or lymphatic system or, more commonly, attached to a cell wall. These antioxidants chemically react with free radical oxidants, neutralizing the oxidant to obviate any oxidation damage to the cell.

Research with diabetic patients revealed that the non-healing in epithelial necrosis of severe diabetics was caused due to “poor circulation” or the inability to get nutrients and disease fighting drugs to the site. More recent studies have observed the lack of stem cells at the site, suggesting this may also be a contributing factor. Clinical studies have shown that the presence of high quantities of cell damaging oxidants destroy pluripotent cells. There is some evidence that topical treatments containing high doses of antioxidants and steroids accelerate the healing of such wounds. The steroids (growth hormones) are thought to be pluripotent cell initiators in this case.

Adult stem cells are found most abundantly in bone marrow. The majority of the stem cells are slowly cycling such that less than about 1% of bone marrow cells are stem cells and of those, only about 10% are cycling at a given time. Stem cell production decreases with age. It is believed that stem cell production decreases in humans by 0.5% with each year beyond age 20.

Increasing pluripotent cell production, especially in elderly persons, is desirable for optimum health. Regeneration of deteriorating tissues prevents malfunctioning and disease. It is equally important to increase the activity of and prolong the life cycle of the pluripotent cells for the regenerative benefits to be realized. Thus, it is desirable to provide a compound that enhances pluripotent cell production, enhances its emission into the bloodstream, increases the activity of the pluripotent cells, prolongs the life cycle of the pluripotent cells, and optimizes the general physiological condition of the subject so the beneficial regenerative effects can be realized by absorption in said stressed tissues.

Disease management is paramount to the health and well being of living organisms. A normal function of the auto immune system is to routinely remove pathogens as they enter our bodies through the various cavities (e.g., mouth, nose, eyes) or penetrate our epithelial system (skin). A second function of our immune system is to attack colonies of pathogens that form in our tissue (e.g., abscesses). Both of these functions are generally performed by the white blood cells in our circulatory system.

There are three general types of white blood cells in our system. Lymphocytes constitute approximately 15-20% of the white blood cells. These lymphocytes contain antibodies, enzymes, and other chemicals used in the destruction of viruses. Monocytes (3-8%) carry antibodies to treat specific diseases. Granulocytes constitute about 75% of the white blood cells in our blood. These are cells that contain nonspecific antibodies, enzymes, and other chemicals for fighting bacterial infections.

The general concentration of the white blood cells is about 8,000 cells per cubic millimeter, but can increase to tens of thousands during an active infection in our body. These cells are produced by the stromal stem cells in the bone marrow. Although an excess of these white blood cells are stored in the bone marrow and other locations, during an active infection, it is necessary for the stromal cells to rapidly reproduce new white blood cells. The rate white blood cells are reproduced is dependent upon the quantity of available stromal cells in the bone marrow.

Granulocytes may be monitored via simple microscopy to identify the life stage of each cell. The stromal cell “daughters,” often referred to as “blast” cells, are very seldom observed in the blood sample since they are generally concentrated in the bone marrow. In blast cells, the nucleus appears as round red ball consuming approximately 90% of the cell when stained with Wright's stain. The nucleus of the next stage, “juvenile cells”, appears as a long curved band-like structure in the cell. The nucleus of “mature cells” appear as three separate segments. The nucleus of final stage and least active “hypersegmented cells” appear as 5-10 segments.

Thus, during an infection where there is an increased need for the granulocytes in the blood system, it is easy to identify the source of the cells. If the cells are coming from a stored source, the amount of mature cells (nucleus appearing as segments) relative to the number of immature cells (nucleus appearing as bands) will increase as compared to the normal ratio. If someone has taken a stimulant to increase the number and/or activity of the stromal cells, the increased production of granulocytes may be accounted for by the higher ratio of immature cells (nucleus appearing as bands) in the blood.

### Components of the Algae Composition

Most antioxidants are obtained by the ingestion of natural products such as fruits and vegetables. An alternative source is from non-pathogenic microbacteria and algae such as AFA, H. pluvialis, Spirulina pacifica, Spirulina plantensis, Dunaliella salina, Chlorella vulgaris and Chlorella pyrenoidosa. They produce antioxidants which appear to be more specific for the reduced destruction of pluripotent cells by oxidants generated from either decomposing tissue, pathogenic bacteria, aging processes and/or normal metabolic processes. The synergism between the cyanobacteria and green algae is an important aspect of this novel algae composition.

The ingredients for a typical novel algae composition according to this invention are set out in proportions of the individual cyanobacteria and algae and may be varied to optimize the formulation to suit the specific needs of the user. It is an important feature of the present invention that it contains a mixture and is not based on a single cyanobacteria or algae. An unexpected synergistic effect has been experienced by the inventor. The combination of a powerful antioxidant with the stem cells provided by AFA, the only known food to increase the body's natural production of, and thus the concentration of, stem cells, are synergistic in combination since the protective aspect of the antioxidants serve to prolong the life cycle of the pluripotent cells, enhance the activity level of the pluripotent cells, and decrease the demand for tissue regeneration.

In one embodiment, about 2 to about 10 grams of cyanobacteria is administrated per day to a subject. In one embodiment, about 1 to about 10 grams of green algae is administered per day to a subject. In one embodiment, the green algae comprises from about 0.01 to about 50 weight per cent of antioxidants, preferably from about 0.01 to about 10.0 weight per cent, more preferably from about 0.01 to about 1.0 weight per cent, and most preferably from about 0.01 to about 0.05 weight per cent. Those antioxidants preserve the pluripotent cells.

Improvements that have been subjectively experienced by the inventor applicant include an improved sense of well being, an improvement in appetite, an increase in endurance and strength during physical exertion, and an increase in vigor and enthusiasm in daily activities. The inventor has observed similar results when the novel composition is administered to his cat and hamsters.

In a preferred embodiment, the novel algae composition of this invention comprises a portion of AFA of from about 2 grams to about 10 grams, preferably about 5 grams.

In a preferred embodiment, the novel algae composition of this invention comprises a portion of Spirulina of from about 2 grams to about 10 grams, preferably about 4 to about 5 grams.

In a preferred embodiment, the novel algae composition of this invention comprises a portion of H. pluvialis comprising of from about 2 milligrams to about 10 milligrams of astaxanthin, preferably about 5 milligrams.

In a preferred embodiment, the novel algae composition of this invention comprises a portion of Chlorella vulgaris and/or Chlorella pyrenoidosa of from about 1 gram to about 4 grams, preferably about 2 grams. In a preferred embodiment, the novel algae composition of this invention comprises a portion of Chlorella vulgaris and/or Chlorella pyrenoidosa of from about 2 grams to about 10 grams, preferably about 4 to about 5 grams.

Spirulina contains antioxidants including antioxidant vitamins beta carotene (provitamin A) C, and E as well as antioxidant minerals selenium, manganese, zinc, copper, iron and chromium; phytonutrients such as phycocyanin, beta carotene, and polysaccharides; probiotics; nutraceuticals; gamma-linolenic acid (GLA); digestible protein; and sulfolipids. As a food, the algae contain all essential amino acids and many of the essential natural sugars. Its therapeutic action includes strengthening the immune system; supporting cardiovascular function and healthy cholesterol levels; improving gastrointestinal and digestive health; enhancing natural cleansing and detoxification; reducing cancer risks with antioxidant protection; inhibiting in vitro replication of HIV-1, Herpes, Influenza, Mumps and Measles; and enhancing stem cell growth. Synergistically, Spirulina works well with Chlorella.

In one embodiment, the microalgae Dunaliella salina is substituted for the H. Pluvailis. In one embodiment, the microalgae Dunaliella salina is added to the H. Pluvailis. Dunaliella salina provides a high source of astaxanathin, beta-CDS, 9-cis-beta-carotene and zeaxanthin.

Effective dosages of beta-CDS and 9-cis-beta-carotene in one embodiment of the novel composition and method of this invention are from about 10 mg to about 20 mg, preferably about 15 mg per day. Effective dosages of zeaxanthin in one embodiment of the novel composition and method of this invention are from about 500 mcg to about 1000 mcg, preferably about 800 mcg per day. Absorportion is increased if the effective dosage is taken with fat sources.

In one embodiment, the novel composition and method of this invention comprises an immunoglobin such as beta-CDS. From about 10 mg to about 20 mg, preferably about 15 mg, of beta-CDS are in one embodiment.

In one embodiment, the novel composition of this invention, and the method of using the same, comprises green algae comprising from about 0.01 to about 50.0 weight percent of micropolysaccharides that bind with environmental toxins.

In one embodiment, the novel composition of this invention, and the method of using the same, comprises green algae comprising an antioxidant selected from the group consisting of zeaxanthin, astaxanthin, 9-CIS-beta carotene and beta-CDS.

Dunaliella comprises 9-cis-beta-carotene, a particularly potent antioxidant that has been shown to be as much as ten times more potent than other beta carotenes at preventing cancers. Dunaliella also comprises beta-CDS, a beta-carotene that has been shown to be an effective anti-cancer agent. Dunaliella also comprises zeaxanthin, a caretenoid that has been shown to be an effective treatment for age related macular degeneration. Dunaliella salina increases the activity of the immune system's macrophages and spleen cells.

There are several species of Chlorella. Those most commonly used in nutritional supplements are Chlorella vulgaris and Chlorella pyrenoidosa. Since Chlorella has a true nucleus, it is a later evolved organism than the cyanobacteria and is symbionic with its primordial ancestor of a similar evolutionary time. Chlorella is much higher in chlorophyll content than other consumable algae varieties (often ranging from 3 to 5% pure natural chlorophyll). Chlorella's tough cell walls are made of complex polysaccharides, which have been shown to stimulate interferon production, exhibit strong anti-tumor activity, and provide super detoxifying properties. Chlorella contains more chlorophyll per gram than any other plant. Chlorella contains protein, carbohydrates, all of the B vitamins, vitamins C and E, amino acids, beta-carotene, rare trace minerals, Chlorella Growth Factor (CGF), chlorophyll, carotenoids, such as astaxanthin, canthaxanthin, flavoxanthin, loraxanthin, neoxanthin, violaxanthin, and echinenone. Therapeutic action of Chlorella includes neutralizing and/or eliminating toxins, pesticides and heavy metals from the body such as DDT, PCB, mercury, cadmium and lead; strengthening the immune system response; rebuilding nerve damage in the brain and nervous system; improving digestion; accelerating healing; protecting against radiation; aiding in the prevention of degenerative diseases; helping in treatment of Candida albicans; relieving arthritis pain; and because of its nutritional content, aiding in the success of numerous weight loss programs. Synergistically, Chlorella works well with Spirulina.

Chlorella, a green algae, contains greater levels of growth factors and clorellan are also present, as well as unique polysaccharide and cell wall compounds. The additional carotenoids in Chlorella work synergistically with beta carotene in cyanobacteria, providing enhanced anti-oxidant protection.

Chlorella has a unique cell chemistry that imparts immune-enhancing properties via the mucopolysaccharides' ability to bind with environmental toxins and carry them safely out of the body. The Chlorella cell wall's complex polysaccharides have been shown to stimulate interferon production and exhibit strong anti-tumor activity.

Chlorella's general rebuilding and detoxifying properties enhance the benefits of the stem cell regenerative processes. Chlorella builds vitality, improves digestion, enhances energy, improves mental clarity, and generally aids with other similar “lack of vitality” disorders.

Similarly, Chlorella's general immune system enhancing properties enhance the benefits of the stem cell regenerative processes. Chlorella stimulates the growth of friendly bacteria, which in turn has the probiotic effect of strengthening gut flora and resisting disease. Chlorella helps protect the body in its fight against both viruses and cancer. It increases macrophage activity and exhibits DNA repair mechanisms. Chlorella increases red blood cells, white blood cells, platelets, and albumin (many people with cancer have a decreased level of albumin). Treatment of viruses and fungi which sap energy, such as candida-overgrowth, Epstein-Barr virus, chronic fatigue immune deficiency syndrome (CFIDS), and AIDS, is advanced by the immune-enhancing qualities of CGF and antiviral effect of chlorophyll.

Chlorella may provide saccharides such as Chlorella polysaccharide Nβ-1.3 glucan and monosaccharides glucuronic acid, arabinose, glucose, and fructose a- and β-anomers. These saccharides work synergistically with the cyanobacteria to enhance homing and cell signaling properties.

Chlorella's Growth Factor helps repair nerve tissues. The GLA content of Spirulina combined with the CGF content of Chlorella act together synergistically: GLA feeds the brain and nervous system with the nutrients while the CGF repairs damaged nerve tissue. This nerve repairing property complements and supports the stem cell regeneration processes.

The nucleic acid found in Chlorella (RNA/DNA) is a descendant of the cyanobacteria and provides a symbiosis (related to their origin in evolutionary time) for directing cellular renewal, growth, and repair. The amount of nucleic acid in the body decreases with age, and insufficient nucleic acid causes premature aging and weakened immunity. This DNA repairing property prevents disease and deterioration of tissues, complementing and supporting the stem cell regeneration processes.

Chlorella contains a greater quantity of fatty acids than Spirulina with approximately 20% constituting the artery-cleansing, omega-3, alpha-linolenic variety. Enhancing circulatory system health promotes the transport of the stem cells to the tissues where they are needed, supporting and enhancing the cyanobacteria's stem cell regeneration processes.

Similarly, well functioning body systems will be more receptive to, and benefit more from, the regeneration processes offered by the additional pluripotent cell production. By combining additional cyanobacteria (e.g., Spirulina) and/or green algae (e.g., Chlorella) components that enhance the general health of the subject and inhibit the harmful effects of disease causing organisms, the regenerative process benefits will be amplified.

In one embodiment, additional cyanobacteria and/or algae components are added for more synergistic effects. Optionally, the cyanobacteria-algae mixture may contain other algae, microbacterial or herbal constituents as desired by the user and/or manufacturer. The user may selectively add algae and/or microbial components and/or herbs that are directed to provide health benefits desirable for the user. It is important, however, that the microbacterial and/or algae components and/or herbs selected do not impair the functionality of the cyanobacteria or green algae.

One commercially available source of cyanobacteria is Now Foods, Bloomingdale, Ill. 60108. One commercially available source of xylitol is Xylosweet™ available through Xlear, Inc., P.O. Box 970911, Orem, Utah 84097 www.xlearinc.com. One commercially available source of Chlorella is “nanized Chlorella concentrate” by Premier Research Labs, Round Rock, Tex. 78664. One commercially available source of xanthan gum is NOW FOODS, Bloomingdale, Ill. 60108 www.nowfoods.com. One commercially available source of astaxanthin is Mera Pharmaceutics, Kailua-Kona, Hi. 96740. One commercially available source of seaweed extract is DHC Corporation, 2-7-1Minami-azabu, Minato-Ku, Tokyo.

### Administration of the Novel Composition

In one embodiment, a novel algae composition comprising a cyanobacteria, such as Aphanizomenon flos aquae (AFA), and a green algae, such as Hoematocaccus Pluvialis (H. Pluvialias), is administered to a subject. In some embodiments, the subject consumes and digests whole cells or extracts of said cyanobacteria and green algae. The cells may be fresh, frozen, freeze-dried, dehydrated, or preserved in some other manner. Preferably, the whole cell extracts are in powdered form.

This effective amount may be administered at a given frequency, such as about once a week, about twice a week, about three times a week, once a day, about twice a day, about three times a day, and the like. Preferably, the effective amount is administered two to four times per day, at least 4 hours apart, over a prolonged period of time, e.g. months or years.

The dietary supplement is preferably administered orally. This may in the form or tablets, liquid, powder, freeze dried product, or liquid suspension. Active ingredients comprise Aphanizonmenon flos-aquae and Heamatococcus pluvialis. Optionally, active ingredients may also include Spirulina pacifica and/or Chlorella vulgaris and Chlorella pyrenoidosa.

The effective amount of the novel algae composition of this invention and frequency of administration may depend on a variety of factors, such as the algae components utilized, the general health of the subject being treated, and the physiological characteristics (e.g., height, age, weight, body fat percentage, metabolism, etc.) of the subject being treated.

For illustrative purposes, ranges of said active ingredients are shown below in weight percentage of the total weight of said active ingredients:

| | |
|---|---|
| Aphanizomenon flos-aquae | 30-70% |
| Haematococcus pluvialis or Dunaliella salina | 30-70% |
| Spirulina pacifica or Spirulina plantenis | 20-30% |
| Chlorella vulgaris or Chlorella pyrenoidosa | 10-20% |

Oral dosage may be administered in a single dosage or multiple dosages over the course of a day. Daily dosage of said active ingredients is to be from about 12 to about 20 grams daily.

It would be difficult to “overdose” on the novel composition because the subject would likely become full and lose any appetite for further ingestion. In one embodiment, it is preferred that Spirulina administered for disease fighting therapy is about 20 grams daily. In one embodiment, it is preferred that Spirulina administered for prophylatic therapy is about 10 grams daily. In one embodiment, it is preferred that Chlorella administered for disease fighting therapy is about 20 grams daily. In one embodiment, it is preferred that Chlorella administered for prophylatic therapy is about 5 grams daily.

Inert ingredients may optionally be added to the novel composition. Depending upon the form the supplement is to be administered, the mixture may contain inert ingredients including, but not limited to, pharmaceutically acceptable carriers, texture enhancers, palatability enhancers, flavorings, sweeteners, surfactants, solid bulk diluents, binders, and preservatives.

Pharmaceutically acceptable carriers function to assist in the administration of the therapeutic moieties. These are well known in the art and include compounds such as water, lipids, salts, surfactants, buffers, thickeners, preservatives, and the like.

One of the advantageous features of the novel compound is its flexibility and versatility for oral consumption by humans. Taste and texture are important characteristics of foods eaten by humans. Different people may have different preferences. The novel compound of this invention may be selectively adjusted by a user to meet the unique user's desirable qualities. By way of example, but not limitation, a user may adjust the amount of texture enhancer (e.g. xanthan gum, guar gum and/or combination thereof and/or taste enhancer (e.g. xylitol) to produce a compound of desirable sweetness and texture. By way of illustration, adding more texture and/or taste enhancer (taking care not to exceed the maximum limits) produces a compound that may be used in the form of a milkshake, pudding, or ice cream topping.

Xylitol is a sweetener that promotes dental health and oral health, has antibacterial properties, and does not produce the harmful health effects of processed and refined sugars. Xylitol has a Glycemic Index of 7, making it particularly well suited for diabetics.

By way of further example, the novel compound may be mixed with soy milk, gelatin, milk powder, milk, berries, chocolate syrup, or juice to increase the variety of oral administration options. This is especially useful for elderly or ill subjects who have depressed appetites.

In one embodiment, the novel algae composition is formed into a powder. Such powder is prepackaged in individual “packet” doses or in a larger bulk container where the user measures spoonfuls or scoopfuls to mix with food products.

In one embodiment, food products containing the novel algae compound are prepared and prepackaged for sale. By way of illustration, but not limitation, such food products include shakes, juices, solid snack foods, candies, desserts, soups, hot drinks, and the like.

The following examples are provided to illustrate particular features of various described embodiments. The scope of the present invention should not be limited to those features exemplified.

In one preferred embodiment, the novel composition comprises active and inert ingredients in powdered form that may be formed into shakes, puddings and the like. In one embodiment, the ingredients are blended with a high speed blender such as a Vita-Mix 5000. This method of blending ensures that algae with relatively strong cellular membranes are made bio-available. In a preferred embodiment containing xylitol as a taste enhancer, the xylitol is added early in the mix sequence to provide a pH range that is friendly to the algae components.

### EXAMPLE 1

### Active Ingredients

| Algae Component | Weight |
|---|---|
| Aphanizomenon flos-aquae | 2 g-10 g |
| Astaxanthin from H. pluvialis or Dunaliella salina | 2 mg-10 mg |
| Spirulina plantensis or Spirulina pacifica | 2 g-9 g |
| Chlorella vulgaris or Chlorella pyrenoidosa | 1 g-4 g |

### EXAMPLE 2

### Active Ingredients

| Algae Component | Weight | |
|---|---|---|
| Aphanizomenon flos-aquae (AFA) | 5 | g |
| Astaxanthin from H. pluvialis or Dunaliella salina | 5 | mg |
| Spirulina pacifica or Spirulina plantensis | 4 | g |
| Chlorella vulgaris or Chlorella pyrenoidosa | 2 | g |

### EXAMPLE 3

### Active Ingredients

| Algae Component | Weight | |
|---|---|---|
| Aphanizomenon flos-aquae (AFA) | 5 | g |
| Astaxanthin from H. pluvialis or Dunaliella salina | 5 | mg |
| Spirulina pacifica or Spirulina placentis | 4 | g |
| Chlorella vulgaris or Chlorella pyrenoidosa | 2 | g |

The therapeutic dosage of astaxanthin may be obtained from about 1 gram to about 10 grams of H. pluvialis or Dunaliella salina. In one embodiment, the astaxanthin is derived from both H. pluvialis or Dunaliella salina.

### EXAMPLE 4

### Active Ingredients

| Algae Component | Weight |
|---|---|
| Aphanizomenon flos-aquae | 2 g-10 g |
| H. pluvialis | 1 g-10 g |
| Spirulina plantensis or Spirulina pacifica | 2 g-9 g |
| Chlorella vulgaris or Chlorella pyrenoidosa | 1 g-4 g |
| Dunaliella salina | 1 g-10 g |

In one embodiment, a texture enhancer, preferably xanthan gum or guar gum, is added. (Xanthan gum also slows the digestive process and enhances the absorption of the cyanobacteria and algae elements.) In another embodiment, a taste or sweetness enhancer, preferably xylitol, is added. In one embodiment, both a taste and texture enhancer are added.

In one embodiment, additional cyanobacteria and/or algae components are added for more synergistic effects. Optionally, the cyanobacteria-algae mixture may contain other algae, microbacterial or herbal constituents as desired by the user and/or manufacturer. The user may selectively add algae and/or microbial components and/or herbs that are directed to provide health benefits desirable for the user. It is important, however, that the microbacterial and/or algae components and/or herbs selected do not impair the functionality of the cyanobacteria or green algae.

In a preferred embodiment, the novel algae composition of this invention comprises a portion of xylose of from about 2 grams to about 36 grams, preferably about 9 grams.

In a preferred embodiment, the novel algae composition of this invention comprises a portion of xanthan and/or guar gum of from about 1 gram to about 10 grams, preferably about 4 grams.

### EXAMPLE 5

### Active and Inert Ingredients

| Algae Component | Weight |
|---|---|
| Aphanizomenon flos-aquae | 2 g-10 g |
| Haematococcus pluvialis | 2 mg-10 mg |
| Spirulina plantensis or Spirulina pacifica | 2 g-9 g |
| Chlorella vulgaris or Chlorella pyrenoidosa | 1 g-4 g |
| Xylose | 2 g-36 g |
| Xanthan or Guar Gum | 1 g-10 g |

### EXAMPLE 6

### Active and Inert Ingredients

| Algae Component | Weight |
|---|---|
| Aphanizomenon flos-aquae | 2 g-10 g |
| Astaxanthin from H. pluvialis or Dunaliella salina | 2 mg-10 mg |
| Spirulina plantensis or Spirulina pacifica | 2 g-9 g |
| Chlorella vulgaris or Chlorella pyrenoidosa | 1 g-4 g |
| Xylose | 2 g-36 g |
| Xanthan or Guar Gum | 1 g-10 g |

### Verification of Utility

Initial verification may be accomplished using traditional laboratory techniques involving double blind studies. One approach would be to use laboratory animals such mice. Mice would be treated with various levels of the active ingredients. Physiological parameters such as mean lifetime, rate of wound healing, pathogenic disease frequency, and organ conditions would be monitored versus untreated animals. Similarly, long term studies could be done with humans, parameters such as sickness frequency, wound healing, blood counts both at healthy state and during illness, physical endurance, and chemical studies to monitor health of various organs, among other test.

In one embodiment, administration of the compound or mixture containing cyanobacteria and a green algae results in the increased production of pluripotent cells from about 1 to about 4 hours following administration. These pluripotent cells will enter the circulatory system, thus increasing the number of circulating pluripotent cells within the subject's body. The percentage increase in the number of circulating pluripotent cells compared to a normal baseline may be about 25%, about 50%, about 100% or greater than about 100% increase as compared to a control. In one embodiment, the control is a base line value from the same subject.

The levels of stem cells available in the circulatory system may be tested according to the teachings of U.S. Pat. No. 6,814,961 disclosing a method to enhance trafficking or homing of stem cells.

As such, those skilled in the art will appreciate that the conception, upon which this disclosure is based, may readily be utilized as a basis for the designing of other structures, methods and systems for carrying out the several purposes of the present invention.

1. A method for enhancing health and disease control in a subject, comprising: administering to a subject a composition comprising a therapeutic dosage comprising green algae and cyanobacteria.

2. The method according to claim 1, where said subject is a mammal.

3. The method according to claim 2, where said subject is a human.

4. The method according to claim 3, wherein from about 1 gram to about 10 grams of said green algae is administered.

5. The method according to claim 4, wherein said green bacteria is administered as whole cells.

6. The method according to claim 3, wherein from about 2 to about 10 grams of said cyanobacteria is administered.

7. The method according to claim 6, wherein from about 1 gram to about 10 grams of said green algae is administered.

8. The method according to claim 6, wherein said cyanobacteria is administered as whole cells.

9. The method according to claim 8, wherein said green bacteria is administered as whole cells, wherein said green bacteria and said cyanobacteria provide increased trace nutrient concentrations.

10. The method according to claim 6, wherein said cyanobacteria enhances the concentration of pluripotent cells.

11. The method according to claim 10, wherein said concentration of pluripotent cells are disposed in an enhanced concentration site.

12. The method according to claim 11, wherein said enhanced concentration site comprises a site selected from the group consisting of a circulatory system, a diseased site, and an injured site.

13. The method according to claim 4, wherein said green algae comprises from about 0.01 to about 50.0 weight percent of antioxidants.

14. The method according to claim 13, wherein said green algae comprises from about 0.01 to about 1.0 weight percent of antioxidants.

15. The method according to claim 14, wherein said green algae comprises from about 0.01 to about 0.05 weight percent of antioxidants.

16. The method according to claim 13, wherein said antioxidants preserve said pluripotent cells.

17. The method according to claim 7, wherein said composition comprises a form selected from the group consisting of a dry powder, liquid suspension and tablet.

18. A method for enhancing the production of pluripotent cells and increasing the longevity of said pluripotent cells in a subject, comprising: administering to a human a composition comprising a therapeutic dosage comprising green algae and cyanobacteria, thereby enhancing pluripotent cell concentration in a subject.

19. The method according to claim 18, wherein from about 2 to about 10 grams per day of said cyanobacteria is administered.

20. The method according to claim 18, wherein from about 1 gram to about 10 grams per day of said green algae is administered.

21. The method according to claim 18, wherein said composition comprises a form selected from the group consisting of a dry powder, liquid suspension and tablet.

22. The method according to claim 19, wherein said cyanobacteria enhances the concentration of pluripotent cells.

23. The method according to claim 20, wherein said green algae comprises from about 0.01 to about 10.0 weight percent of antioxidants.

24. The method according to claim 23, wherein said green algae comprises from about 0.01 to about 1.0 weight percent of antioxidants.

25. The method according to claim 23, wherein said antioxidants preserve said pluripotent cells.

26. The method according to claim 18, wherein said composition comprises about 5 grams of Aphanizomenon flos-aquae, from about 4 grams to about 5 grams of Spirulina, and about 2 grams of Chlorella.

27. The method according to claim 20, wherein said green algae comprises from about 0.01 to about 50.0 weight percent of mucopolysaccharides.

28. The method according to claim 27, wherein said mucopolysaccharides bind with environmental toxins.

29. A composition for enhancing health and disease control in humans, comprising: a therapeutic dosage comprising green algae and cyanobacteria.

30. The composition of claim 29, wherein said green algae comprises a green algae selected from the group consisting of Haematoccus pluvialis, Chlorella vulgaris, Chlorella pyrenoidosa, Dunaliella salina or a combination thereof

31. The composition of claim 29, wherein said cyanobacteria comprises a cyanobacteria selected from the group consisting of Aphanizomenon flos-aquae, Spirulina pacifica, Spirulina plantesis or a combination thereof.

32. The composition of claim 29, wherein said therapeutic dosage comprises from about 30 to about 70 weight percent of said green algae.

33. The composition of claim 29, wherein said therapeutic dosage comprises from about 30 to about 70 weight percent of said cyanobacteria.

34. The composition of claim 29, wherein said composition further comprises pharmaceutically acceptable carriers.

35. The composition of claim 29, wherein said composition further comprises a palatability enhancer.

36. The composition of claim 29, wherein said composition comprises a form selected from the group consisting of a dry powder, liquid suspension and tablet.

37 The composition of claim 32, wherein said green algae comprises from about 0.01 to about 10.0 weight percent of antioxidants.

38. The composition of claim 37, wherein said green algae comprises from about 0.01 to about 1.0 weight percent of antioxidants.

39. The composition of claim 37, wherein said 0.01 to about 10.0 weight per cent of antioxidants comprises at least one antioxidant selected from the group consisting of zeaxanthin 9-cis-beta-carotene and beta-CDS.

40. The composition of claim 37, wherein said composition comprises from about 2 milligrams to about 3.75 milligrams of astaxanthin.

41. The composition of claim 37, wherein said composition comprises about 5 milligrams of astaxanthin.

42. The composition of claim 39, wherein said composition comprises from about 500 micrograms to about 1000 micrograms of zeaxanthin.

43. The composition of claim 42, wherein said composition comprises about 800 micrograms of zeaxanthin.

44. The composition of claim 39, wherein said composition comprises from about 10 milligrams to about 20 milligrams of 9-cis-beta-carotene.

45. The composition of claim 44, wherein said composition comprises about 15 milligrams of 9-cis-beta-carotene.

46. The composition of claim 39, wherein said composition comprises from about 10 milligrams to about 20 milligrams of beta-CDS.

47. The composition of claim 46, wherein said composition comprises about 15 milligrams of beta-CDS.

48. The composition of claim 29, wherein said composition comprises about 5 grams of Aphanizomenon flos-aquae, about 4 grams to about 5 grams of Spirulina pacifica, and about 2 grams of Chlorella vulgaris.

49. The composition of claim 29, wherein said composition further comprises an immunoglobulin.

50. The composition of claim 49, wherein said immunoglobulin comprises beta-CDS.

51. The composition of claim 29, wherein said composition further comprises an L-selectin ligand.

52. The composition of claim 29, wherein said composition comprises Haematoccus pluvialis, Aphanizomenon flos-aquae and Dunaliella salina.

53. The composition of claim 52, wherein said composition further comprises Chlorella vulgaris and Spirulina pacifica.
